(19) United States
(12) Patent Application Publication
(10) Pub. No.: US 2005/0277861 A1
(43) Pub. Date: Dec. 15, 2005

(54) Fistula or graft sleeve-like apparel

(76) Inventor: Maggie Helen Anthony, Voorhees, NJ (US)

Correspondence Address: Maggie Helen Anthony, 296-2 Echelon Road, Voorhees, NJ, 08043, US

(21) Appl. No.: 10/855,470

(22) Filed: May 28, 2004

(51) Int. Cl. A61F 13/06; A61M 25/00

(52) U.S. Cl. 602/62

(57) ABSTRACT

A sleeve-like apparel which includes Lycra material, or a heavy blend of Lycra material having a variety of brilliant colors, or neutral colors and adornments. Flexibility of material's top, bottom, length and width of sleeve-like apparel will fit circumference of user's upper or lower arm and aid in forming material to configuration of user's upper or lower arm without pressuring or harming user's Fistula, or Graft vascular access. All the while sleeve-like apparel will conceal protruding Fistula, Graft access, related six to seven inch surgical scar and multiple needle marks located on top of the Fistula or Graft; thereby, eliminating the need to conceal Fistula, or Graft, surgical scar, needle marks with long sleeve clothing during hot, humid weather conditions, climates, or seasons. A particular feature of the invention is that once the sleeve-like apparel has been adjusted to user's desired optimum position, the sleeve-like apparel will remain in adjusted position until purposely changed, or taken off user's arm.

### CROSS-REFERENCE TO RELATED APPLICATIONS

NOT APPLICABLE

### STATEMENT REGARDING FEDERALLY SPONSORED RESEARCH OR DEVELOPMENT

NOT APPLICABLE

### REFERENCE TO SEQUENCE LISTING, A TABLE, OR A COMPUTER PROGRAM LISTING COMPACT DISC APPENDIX

NOT APPLICABLE

### BACKGROUND OF THE INVENTION

### FIELD OF INVENTION

The present invention relates in general to a cosmetic sleeve apparel, in particular to a cosmetic sleeve-like apparel for permanent dialysis patients who have either Fistulas, or Graft vascular accesses located on her, or his upper or lower arm.

Tens of thousands of middle-aged people who are still employed, senior citizens who are active in our communities and young people who are just starting life-long goals are diagnosed with End-Stage Renial Disease (kidney Failure) each year in the United States. As a result, theses patients have to receive dialysis (removing waste material from blood) to sustain their lives. Short term dialysis is achieved with the use of a catheter that's inserted into a large vein just underneath the area of the collar bone, thus wearing short sleeve and sleeveless clothing doesn't usually present a problem because the catheter is easily concealed by clothing around the collar bone area. As time progresses, a large percentage of these dialysis patients generally require permanent dialysis, or a kidney transplant to sustain their lives.

Currently, more than 216,000 Americans have to have on-going, or permanent dialysis without the use of a catheter which requires a procedure that connects an artery with a vein generally in the upper, or lower arm underneath the skin. These two main accesses for dialysis are called a Fistula or Graft vascular access.

Over several weeks, the Fistula or Graft vascular access mature. consequently, causing the artery/vein to bulge and develops even larger as time progresses. A mature access is the desired effect needed to allow the two large gage needles to be inserted into the Fistula or Graft. two to three times each week to remove waste intravenously. As a result, the constant insertion of needles into the access leave needle marks on top of the Fistula or Graft. Additionally, there is a six to seven inch surgical scar relevant to the actual procedure for the Fistula or graft that's located in close proximity to the Fistula or Graft access.

Situations exist during cool or cold weather conditions, or climates when these dialysis patients can easily conceal their Fistula, or Graft vascular accesses, multiple needle marks, and related surgical scar with ordinary long sleeve garments. There are some dialysis patients; however, who elect to wear weather or climate appropriate garments, short sleeves and sleeveless garments without concealing the Fistula, Graft, needle marks and surgical scar; nonetheless, find themselves engaged in unwarranted conversations about her or his Fistula, Graft, needle marks and surgical scar.

Even if a patient receives a kidney transplant and no longer needs dialysis to sustain her or his life, the surgical scar, protruding artery/vein and needle marks remain visible for several months or years before they diminish in color or size. Thus how to wear weather and climate appropriate clothing with dignity still remain an issue.

### BRIEF SUMMARY OF THE INVENTION

Accordingly, the inventor of the present invention recognized a need for a sleeve-like apparel for long-term dialysis patient's upper arm, or forearm that functions like an arm accessory to conceal area of arm that's grossly effected by either Fistula, or Graft vascular access, related surgical scar, needle marks, and yet, coordinates with user's short sleeve, or sleeveless garment; thereby, eliminating the patient's need to wear inappropriate weather, climate, and seasonal clothing and bandages to conceal area of arm effected by Fistula, or Graft vascular access. It will also minimizes the wearer's/user's compulsion and displeasure in having to repeatedly explain to strangers, co-workers, friends and relatives the relevant medical history of the uncovered, disfigured portion of the arm that's effected by the Fistula, or Graft vascular access.

An aspect of the invention involves the tubular-like shape and hollow opening at the top of the sleeve-like apparel to allow wearer's/user's arm to be inserted when the sleeve is put on and bottom of sleeve that has an adaptable opening to receive the remaining portion of arm and hand of wearer. Because of the elasticity and light-weight of sleeve's Lycra material, sleeve-like apparel will fit comfortably overall as it conforms to the natural contours of wearer's arm while effectively covering bulky Fistula, or Graft access, related scar and large gage needle marks.

Another aspect of the invention is that the apparel is an invention for females and males and can fit either right arm or left arm effected by Fistula or Graft vascular access.

### BRIEF DESCRIPTION OF THE SEVERAL VIEWS OF THE DRAWINGS

NOT APPLICABLE

### DETAILED DESCRIPTION OF THE INVENTION

The measurements for material of said sleeve-like apparel for a user's upper or lower arm will vary in length and width, depending on size of user's upper or lower arm; however, variation of sizes would be constructed in the same manner and have the same function as the medium sized sleeve-like apparel described below. Sleeve-like apparel will range in sizes from extra large to small. The example below is an illustration of measurements for a medium size sleeve-like apparel for a user's upper arm and medium size sleeve-like apparel for a user's lower arm. Both examples of the sleeve-like apparel for user's upper arm and lower arm include the description of the top opening and bottom opening of the sleeve-like apparel.

For upper arm, sleeve-like apparel comprises a piece of Lycra material, or blend of Lycra material 9½ inches in length and 11½ inches in width. Sleeve-like apparel also comprises a ½ inch hem thereon the underside of top, or width of said material of sleeve-like apparel and having a ½ inch hem thereon the underside of bottom, or width of bottom of said sleeve-like apparel. Left side of said material's length on the underside is sewn to the right side of said material's length on the underside comprising the seam down the center, forming the tubular-like shape of the sleeve-like apparel.

For lower arm, sleeve-like apparel comprises a piece of Lycra material, or blend of Lycra material 9 inches in length and 7½ inches in width. Sleeve-like apparel also comprises ½ inch hem thereon the underside of the top of sleeve-like apparel, or width of top said material and having a ½ inch hem thereon on the underside of said sleeve-like apparel, width of bottom of said material. Left side of said material's length on the underside is sewn to the right side of said material's length on the underside comprising seam down the center, forming the tubular-like shape of the sleeve-like apparel.

Tubular-like shape of said sleeve-like apparel for user's upper arm has a hollow opening at the top similar to a hollow opening of a detached sleeve of an ordinary sleeved garment. The top opening and overall width of sleeve-like apparel is approximately 11½ inches in circumference to allow user to insert her or his arm through the top opening, or top of said sleeve-like apparel, pulling the sleeve up the arm three to four inches below the shoulder to cover fistula or graft vascular access on upper arm. Bottom of the upper sleeve-like apparel has an opening like the hollow opening of a detached sleeve of an ordinary sleeved garment. The bottom of upper sleeve-like apparel is approximately 11½ inches in circumference to allow user's elbow to go through the bottom opening of sleeve-like apparel where sleeve-apparel ends one to two inches below the user's elbow.

Tubular-like shape of said sleeve-like apparel for user's lower arm has a hollow opening at the top similar to a hollow opening of a detached sleeve of an ordinary sleeved garment.

The top opening and overall width of sleeve-like apparel is approximately 7½ inches in circumference to allow user to insert her or his hand and arm through the opening or top of said sleeve-like apparel, pulling up top of sleeve-like apparel one to two inches below her or his elbow.

Sleeve-like apparel for lower arm has a hollow opening at the bottom like the hollow opening of a detached sleeve of an ordinary sleeved garment. The bottom opening of lower sleeve-like apparel is approximately 7½ inches in circumference to allow user to pull her or his hand and arm through the opening of sleeve-like apparel and out through the bottom opening of the sleeve-like apparel and said bottom of sleeve-like apparel ending about one to two inches before her or his wrist.

Because of the elasticity of said material of sleeve-like apparel, top and bottom will comfortably fit circumference of user's lower arm while elasticity of material lengthwise and overall elasticity of width will conform to the shape of user's lower arm leaving user's hand free from coverage of the sleeve-like apparel without pressuring, or harming user's fistula, or graft vascular access.

Once said sleeve-like apparel is positioned on user's upper or lower arm which ever is effected by fistula or graft vascular access, sleeve-like apparel will remain in adjusted position as it conceals protruding fistula or graft access, as well as six to seven inch surgical scar and conceal multiple needle marks on fistula or graft access resulting from large gage needles that are inserted into the fistula or graft multiple times each week.

1. Sleeve-like apparel comprising Lycra material for concealing the unsightly and irregular bulging of mature fistula or graft vascular access that is generally located on dialysis patients upper or lower arm, while sleeve-like apparel simultaneously conceal related six to seven inch discolored surgical scar that's normally located two to three inches adjacent to fistula or graft vascular access on patient's upper arm, or lower arm. Moreover, sleeve-like apparel concurrently conceal needle marks visible on top of fistula or graft access that is the result of either two seventeen gage needles, two sixteen gage needles, or two fifteen gage needles that are inserted into dialysis patients fistula or graft, two to three times each week to allow the removal of waste material from the blood intravenously.

2. A sleeve-like apparel as defined in claim 1 wherein apparel is for a user's upper right arm, upper left arm, lower right arm, or lower left am, whichever arm, or part of arm that is effected by a fistula, or graft vascular access, surgical scar and large gage needle marks.

3. A sleeve-like apparel as defined in claim 1 where said sleeve-like apparel for either upper, or lower arm will conform to the natural contours of user's upper or lower arm and remain in optimum adjusted position until purposely changed or taken off user's arm.

4. A sleeve-like apparel as defined in claim 1 wherein said material of sleeve-like apparel will be made available in an assortment of solid colors, combination of colors and can include clothing stickers for sporting events, business and social occasions and include a variety of adornments to coordinate with user's overall attire.

5. a sleeve-like apparel as defined in claim 1 wherein sleeve-like apparel is an apparel for both females and males, unless adornments are characteristically female or male.

6. A sleeve-like apparel as defined in claim 1 and illustration in description of invention wherein sleeve-like apparel will be made available in a variety of sizes, extra large, large, medium, small, etc.
